(19) United States
(12) Patent Application Publication
(10) Pub. No.: US 2021/0275577 A1
(43) Pub. Date: Sep. 9, 2021

(54) MOLECULAR HYDROGEN-CONTAINING COMPOSITION FOR REGULATING INTRACELLULAR SIGNAL TRANSDUCTION

(71) Applicants: MiZ Company Limited, Kanagawa (JP); Osaka University, Osaka (JP)

(72) Inventors: Fumitake SATOH, Kanagawa (JP); Bunpei SATO, Kanagawa (JP); Shinichi HIRANO, Kanagawa (JP); Ryosuke KUROKAWA, Kanagawa (JP); Yusuke ICHIKAWA, Kanagawa (JP); Hiroshi MATSUURA, Osaka (JP); Kentaro SHIMIZU, Osaka (JP); Hiroshi OGURA, Osaka (JP); Takeshi SHIMAZU, Osaka (JP)

(73) Assignees: MiZ Company Limited, Kanagawa (JP); Osaka University, Osaka (JP)

(21) Appl. No.: 17/191,417

(22) Filed: Mar. 3, 2021

(30) Foreign Application Priority Data
Mar. 9, 2020 (JP) 2020-056838

(51) Int. Cl. A61K 33/00; A61K 9/00; A61P 31/04; A61P 3/10; C01B 3/02

(52) U.S. Cl. CPC A61K 33/00; A61K 9/0053; A61K 9/007; C01B 3/02; A61P 31/04; A61P 3/10; A61K 9/0019

(57) ABSTRACT

The present invention provides a composition for regulating intracellular signal transduction. More specifically, the present invention provides a composition for prevention and/or improvement of a symptom associated with intracellular signal transduction in a subject, comprising molecular hydrogen as an active ingredient.

### RELATED APPLICATIONS

This application claims priority to Japanese Patent Application No. 2020-056838, filed on Mar. 9, 2020, the entire content of which is incorporated herein by reference.

### BACKGROUND

### 1. Field of the Invention

The present invention provides a molecular hydrogen-containing composition for regulating intracellular signal transduction in subjects.

### 2. Description of the Related Art

Molecular hydrogen is known to reduce inflammation by eliminating hydroxyl radicals and peroxynitrite, which are reactive oxygen species generated in intracellular organelles such as mitochondria and the nucleus or in cells.

Previously, the Faculty of Medicine, Osaka University and MiZ Company Limited (Ofuna, Kanagawa) pointed out that molecular hydrogen was able to show an anti-oxidative reactivity, which reduces oxidative stress caused by reactive oxygen species, suppress bacterial translocation, and improve enteric bacterial flora when supersaturated physiological saline containing hydrogen (7 ppm) was orally given (15 ml/kg per day for 7 days) by gavage to cecal ligation and puncture (CLP) model mice (Japanese Patent No. 6601851). However, because hydrogen is a smallest diatomic molecule having diffusibility, and hydrogen taken up into the body has a property of permeating cells by diffusion in the body and finally being discharged from the body to the outside the body easily by exhalation, flatus, and the like, the minimum amount required for hydrogen to act effectively varies greatly depending on the disease and the organ (Yuh Fukai, “Hydrogen molecule is amazing: Front line of life science and medical effect,” Kobunsha Paperback). Additionally, since hydrogen administration methods have not been established in detail unlike general drugs, and it is known that the medical effect varies greatly depending on the administration method (e.g., oral administration, inhalation, drip infusion, intraperitoneal administration), the hydrogen administration method needs to be examined in detail for each disease or organ (Yuh Fukai, “Hydrogen molecule is amazing: Front line of life science and medical effect,” Kobunsha Paperback). However, because intracellular signal transduction induced by administration method of hydrogen is unknown for each organ, intracellular signal transduction has not been controlled by a hydrogen administration method or the like to an extent that a treatment strategy can be established.

Under such circumstances, the object of the present invention is to enhance the efficiency of the control of blood glucose levels and the prevention and improvement of inflammatory diseases based on a treatment strategy to regulate intracellular signal transduction by inhalation of molecular hydrogen.

### SUMMARY

That is, the present invention encompasses the following characteristics:

(1) A composition for regulating intracellular signal transduction by inhalation of the composition by a subject, comprising molecular hydrogen as an active ingredient.

(2) The composition according to (1), wherein the intracellular signal transduction is glycolysis, gluconeogenesis, and/or carbohydrate metabolism, and/or inflammatory signal transduction in an organ.

(3) The composition according to (2), wherein a gene related to the gluconeogenesis is one or more genes selected from the group consisting of PFKB1, PGAM2, G6PC, and PCK1.

(4) The composition according to (2), wherein a gene related to the glycolysis and the gluconeogenesis is one or more genes selected from the group consisting of PGAM2, ADH4, G6PC, GCK, and PCK1.

(5) The composition according to (2), wherein a gene related to the glycolysis and the carbohydrate metabolism is one or more genes selected from the group consisting of PDK2, PFKB1, PCDH12, ST6GALNAC6, PGAM2, PTK2B, ADRA1B, ADRB3, C1QTNF, ALDH1A1, ALDH1B1, PRKAG2, KLB, SLC2A2, SLC2A5, PPP1R3C, G6PC, SORD, B4GALT4, GALM, GCGR, GCK, PTH1R, GPD1L, NEU2, DHDH, ANGPTL3, CA5A, GPD1, EPM2A, GNE, PPP1R3B, RBP4, HAGH, and PCK1.

(6) The composition according to (2), wherein the organ is the liver, the lung, and/or the intestine.

(7) The composition according to (1) or (6), wherein a pathway of the inflammatory signal transduction is acute phase response signaling in the liver and/or the intestine, LPS/IL-1 mediated inhibition of RXR function in the liver and/or the intestine, IL-6 signaling in the liver and/or the intestine, STAT3 pathway in the intestine, HMGB1 signaling in the intestine, or interferon signaling in the lung.

(8) The composition according to (7), wherein a gene related to the inflammatory signal transduction pathway is one or more genes selected from the group consisting of genes coding for Cd14 antigen in the liver, TGF-beta activated kinase 1/MAP3K7 binding protein 1 in the liver, suppressor of cytokine signaling 1 in the liver, interleukin 1 receptor antagonist in the liver, interleukin 6 receptor alpha in the intestine, tumor necrosis factor receptor superfamily member 9 in the intestine, interleukin 1 receptor-like 1 in the intestine, suppressor of cytokine signaling 1 in the intestine, lipopolysaccharide binding protein in the intestine, interleukin 10 in the lung, and suppressor of cytokine signaling 1 in the lung.

(9) The composition according to (8), wherein, in a transduction pathway in the liver involving the gene related to the inflammatory signal transduction pathway, a signal is transmitted from IL-2 and/or IL12 outside a cell, via CD11 and/or TLR2 in the plasma membrane of the cell, further via NFkBIA in the cytoplasm, and via NFkB, STAT3, NFkB1, STAT1, STAT4, STAT6, STAT6A, SP1, RCLA, and/or GEBPB in the nucleus of the cell.

(10) The composition according to (8), wherein, in a transduction pathway in the intestine involving the gene related to the inflammatory signal transduction pathway, a signal is transmitted from IL6, IL1B, TNF, and/or IFN6 outside a cell, via NFkBIA in the cytoplasm, and further via NFkB, STAT3, RELA, JUN, TP53, STAT1, FOS, REL, Ap1, and/or MYC in the nucleus of the cell.

(11) The composition according to (8), wherein, in a transduction pathway in the lung involving the gene related to the inflammatory signal transduction pathway, a signal is transmitted from IL10, IL15, Ige, TNF, and/or IFNG outside a cell, via STAT5a/b and/or NFkBIA in the cytoplasm, and further via STAT3, NFkB, STAT1, NFkB1, JUN, CEBPB, and/or RELA in the nucleus of the cell.

(12) The composition according to any one of (1) to (11), which is a gas or a liquid comprising the molecular hydrogen.

(13) The composition according to any one of (1) to (12), wherein the gas comprising the molecular hydrogen has a hydrogen concentration of higher than zero (0) and not higher than 18.5% by volume.

(14) The composition according to any one of (1) to (13), wherein the liquid comprising the molecular hydrogen has a hydrogen concentration of 3 ppm to 10 ppm.

(15) The composition according to any one of (1) to (14), wherein the subject is a mammalian including a human.

(16) The composition according to any one of (1) to (15), which is produced by using a hydrogen gas generating apparatus or a hydrogen water generating apparatus.

(17) The composition according to any one of (1) to (16), wherein a disease controllable by the regulation of intracellular signal transduction is one or more diseases selected from the group consisting of sepsis, inflammatory bowel diseases (ulcerative colitis, Crohn's disease, intestinal Bechet's disease), pneumonia (bacterial pneumonia, viral pneumonia, fungal pneumonia, interstitial pneumonia, allergic pneumonia), COPD, hepatitis (viral hepatitis, alcoholic hepatitis, NAFLD, drug-induced hepatitis, autoimmune hepatitis), diabetes mellitus, and complications of diabetes mellitus.

(18) The composition according to any one of (1) to (17), wherein, based on the intracellular signal transduction in each of the organs, the intracellular signal transduction is regulated by an administration method selected from the group consisting of inhalation of a hydrogen gas, oral administration of hydrogen water, and drip infusion and/or intraperitoneal administration of hydrogen-containing physiological saline.

(19) A system for deriving a treatment method and/or a treatment strategy for prevention or improvement of the disease according to claim 17 using the composition according to the above (1) to (18).

### Advantageous Effects of Invention

It has been demonstrated that the present invention can regulate an inflammatory signal in each organ in subjects and improve even a refractory disease by administering the composition according to the present invention to a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows survival rates in cecal ligation and puncture (CLP) model mice for a group of inhaling a hydrogen gas for 2 hours per day and a group of continuously inhaling the gas for 24 hours per day;

FIG. 2 shows comparisons of changes in serum IL-6 and TNF-α levels and blood glucose levels in cecal ligation and puncture (CLP) model mice among a group of inhaling hydrogen for 24 hours, a sham treatment group, and a control group;

FIG. 3 shows Venn diagrams showing gene levels changing −2-fold or more and 2-fold or more for each organ of the liver, the intestine, and the lung and overlaps thereof;

FIGS. 4A-4C show negative z-scores, which indicate inactive pathways, listed in the order of the p value for canonical pathway analysis analyzed by Ingenuity Pathway Analysis (IPA);

FIG. 5 shows comparisons of gene transcription intensity in each organ of the lung, the intestine, and the liver between the hydrogen group and the control group;

FIG. 6 shows a signal transduction pathway related to CD14 in the liver which is regulated by hydrogen;

FIG. 7 shows a signal transduction pathway related to IL-6 in the intestine which is regulated by hydrogen;

FIG. 8 shows a signal transduction pathway related to IL-10 in the lung which is regulated by hydrogen;

FIG. 9 is a list of genes related to gluconeogenesis increased by inhalation of a hydrogen gas;

FIG. 10 is a list of genes related to carbohydrate metabolism/gluconeogenesis metabolism increased by inhalation of a hydrogen gas; and

FIG. 11 is a list of genes related to carbohydrate metabolism increased by inhalation of a hydrogen gas.

### DETAILED DESCRIPTION OF THE PREFERRED EMBODIMENTS

The present invention will be described in more detail below.

### 1. Composition for Regulating Intracellular Signal Transduction

The present invention provides a composition for regulating intracellular signal transduction, containing molecular hydrogen as an active ingredient.

In the present specification, the term “intestine” refers to the digestive organ involved in food digestion, which begins at the oral cavity, continues through the pharynx, esophagus, stomach, small intestine (duodenum, jejunum, and ileum), and large intestine, and ends at the anus.

In the present specification, the term “lung” refers to the respiratory organ involved in breathing, including the trachea, upper lobe, middle lobe, lower lobe, bronchi, pleura, and diaphragm, as well as cells, blood vessels, tissues, and so forth that constitute them.

In the present specification, the term “liver” refers to tissues classified into right lobe, left lobe, quadrant lobe, and caudate lobe in appearance by falciform ligament of the liver, round ligament of the liver, and venous ligament, as well as cells, blood vessels, portal vein, tissues, and so forth that constitute them.

In the present specification, the term “acute phase response signaling” refers to acute phase response signaling involving interleukin 6 (IL-6).

In the present specification, the term “subject” includes mammalians such as primates including humans, pet animals such as dogs and cats, and ornamental animals such as zoo animals. Preferred subjects are humans.

In the present specification, the term “disease associated with inflammatory signal transduction in an organ” includes diseases associated with bacterial inflammation, such as bacterial enteritis.

In the present specification, the term “disease associated with intracellular signal transduction involved in glycolysis, gluconeogenesis, and/or carbohydrate metabolism” includes diabetes mellitus and complications of diabetes mellitus.

In the present specification, the term “refractory disease” refers to a disease which is impossible or difficult to improve or resolve with current medicine or any drugs manufactured and marketed in the pharmaceutical industry, and for which treatment methods have not been established because of low evidence.

In the present specification, the term “system for prevention or improvement of a disease” refers to a system for analyzing data on intracellular signal transduction obtained by the present invention and deriving guidance to select an appropriate treatment method for prevention or improvement of diseases associated with the present invention using a computer, a computer network, artificial intelligence, and the like, and is composed of a network interface card, a repeater, a hub, a bridge, a switching hub, a router, a program, a power supply unit, and so forth.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen (i.e., the composition of the present invention) is higher than zero (0) and not higher than 18.5% by volume, for example, 0.5% to 18.5% by volume, preferably 1% to 10% by volume, for example, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, and the like. In the present invention, higher hydrogen gas concentrations (but below the explosion limit) or higher daily hydrogen doses tend to be associated with greater effects of regulating intracellular signal transduction. The hydrogen inhalation time per day is preferably two hours or longer, more preferably six hours or longer, yet more preferably 12 hours or longer, most preferably 24 hours. The inhalation may be either continuous inhalation or inhalation with intervals.

In a usual hydrogen gas inhalation therapy, a hydrogen gas exhibits an effect of improving a disease only at a high concentration of 66% or 99%. In the present invention, however, hydrogen is preferably added to the composition of the present invention and administered to subjects such as humans with conditions safe for subjects, and a sufficient effect of improving a disease can be exhibited by regulating intracellular signal transduction even at low hydrogen concentrations of higher than 0 (zero) and not higher than 18.5%.

When a gas other than hydrogen gas is air, the air concentration is in the range of, for example, 81.5% to 99.5% by volume.

When a gas other than hydrogen gas is a gas containing oxygen gas, the oxygen gas concentration is in the rage of, for example, 21% to 99.5% by volume.

As another main gas, for example, nitrogen gas can be further added.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1.2 to 9 ppm, for example, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, 5 to 7 ppm, and 7 to 10 ppm.

In the present invention, higher dissolved hydrogen concentrations (but below the explosion limit) or higher daily hydrogen doses tend to be associated with greater effects of regulating intracellular signal transduction.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/technology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

A gas containing molecular hydrogen or a liquid containing molecular hydrogen (e.g., water [e.g., purified water, sterilized water], physiological saline, drip infusion solution) prepared using the above-mentioned apparatuses or devices can be administered orally or parenterally to subjects preoperatively, perioperatively, or postoperatively.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas is inhaled from the mouth or the nose via a nasal cannula or a mask-like device covering the mouth and the nose, transported to the lungs, and delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 2 hours per dose. Because a longer inhalation time per day is associated with a greater improving effect, the gas can be administered over, for example, three hours or longer, four hours or longer, five hours or longer, six hours or longer, eight hours or longer, 12 hours or longer, 18 hours or longer, or further longer. Additionally, when a gas containing molecular hydrogen is administered by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).

### 2. Method for Regulating Intracellular Signals

The present invention further provides a method for regulating intracellular signals in a subject receiving the above-mentioned composition containing molecular hydrogen as an active ingredient.

The dose, the administration method, and the like of the composition containing molecular hydrogen are as described in the above 1.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Or, in the method of the present invention, for example, 200 to 500 ml per dose for intravenous administration or, for example, 500 to 1000 ml per dose for oral administration of a liquid containing molecular hydrogen at a concentration of, for example, 1 to 10 ppm, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, 5 to 7 ppm, or the like, preferably 3 to 10 ppm, 4 to 10 ppm, 5 to 10 ppm, 5 to 9 ppm, 5 to 8 ppm, 5 to 7 ppm, or the like can continue to be administered to subjects for, for example, 0.5 to three months or longer, four to seven months or longer, one to three years or longer.

Regulation of intracellular signal transduction of the invention according to present application enables a treatment strategy using hydrogen (inhalation of a hydrogen gas, oral administration of hydrogen water, intraperitoneal administration of hydrogen water or hydrogen-containing physiological saline) for improvement of the following diseases associated with inflammation in the intestine, the lung, and the liver: Sepsis, inflammatory bowel disease (ulcerative colitis, Crohn's disease, intestinal Bechet's disease), pneumonia (bacterial pneumonia, viral pneumonia, fungal pneumonia, interstitial pneumonia, allergic pneumonia), COPD, hepatitis (viral hepatitis, alcoholic hepatitis, NAFLD, drug-induced hepatitis, autoimmune hepatitis), diabetes mellitus, complications of diabetes mellitus, and the like.

Based on the intracellular signal transduction data obtained by the present invention, it can be inferred that, for example, an effect of improving a disease associated with the lung or the liver may be obtained more easily by inhalation of a hydrogen gas than administration of hydrogen water or hydrogen-containing physiological saline, and an effect of improving a disease associated with the intestine may be obtained more easily by administration of hydrogen water and oral administration or intraperitoneal administration of hydrogen-containing physiological saline than inhalation of a hydrogen gas.

Additionally, an effect of improving diabetes mellitus and complications of diabetes mellitus is obtained more easily by inhalation of a hydrogen gas than administration of hydrogen water or hydrogen-containing physiological saline because genes related to glycolysis and gluconeogenesis are upregulated by inhalation of a hydrogen gas.

A treatment method and a treatment strategy based on the data on intracellular signal transduction according to the present invention may be derived using a computer, a computer network, artificial intelligence, and the like.

Further, therapeutic agents used for regulation of intracellular signal transduction may be used in combination, if necessary, in the method of the present invention. Combination use of therapeutic agents can increase the precision of regulation of intracellular signal transduction.

### EXAMPLE

The present invention is explained more specifically with reference to the following example. However, the example is not intended to limit the scope of the present invention.

### Example 1

### <Methods>

Cecal ligation and puncture (CLP) or sham surgery were performed on 9-week-old C57BL/6 male mice. Then, the CLP model mice were forced to inhale a 7% hydrogen gas. The inhalation time were two hours per day for one group and continuous 24 hours for the other group. The mice were housed in a chamber in which the concentration of a hydrogen gas could be maintained by a constant supply of a hydrogen gas from a gas generating apparatus (MHG-2000α manufactured by MiZ Company Limited), and forced to inhale the hydrogen gas therein. The mice in the control group were forced to inhale air not containing hydrogen. Mice were bred under a hydrogen gas atmosphere for one week, and the survival rate was evaluated. The serum inflammatory cytokine levels were evaluated using a Quantikine ELISA Kit (INFO) at 24 hours after CLP. The liver, the intestine, and the lung of mice were evaluated by RNA sequencing after inhalation of a hydrogen gas. Data were analyzed using Ingenuity Pathways Analysis (IPA: Qiagen Inc.). Additionally, expression of inflammatory genes in the intestine was investigated by RT-PCR.

### <Results>

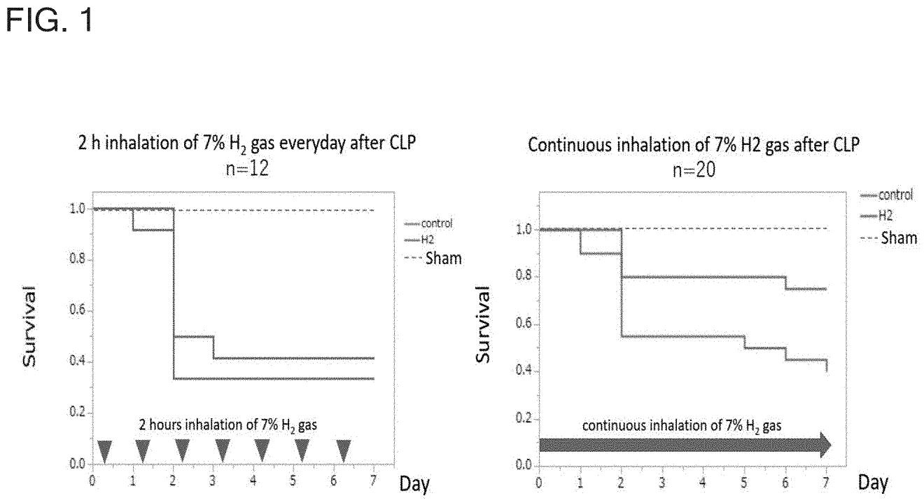

The survival rate of sepsis mice receiving continuous inhalation over 24 hours for 7 days improved significantly compared with the control group (inhalation of air not containing hydrogen) (75% vs. 40%, p<0.05, n=20 vs. 20) [FIG. 1]. When the continuous inhalation time per day was one hour, no difference from control was observed, but when the continuous inhalation time was two hours, differences started to be observed in the survival rate between the hydrogen group and the control group. The survival rate in the hydrogen group improved as the continuous inhalation time was increased to five hours, 12 hours, and 24 hours.

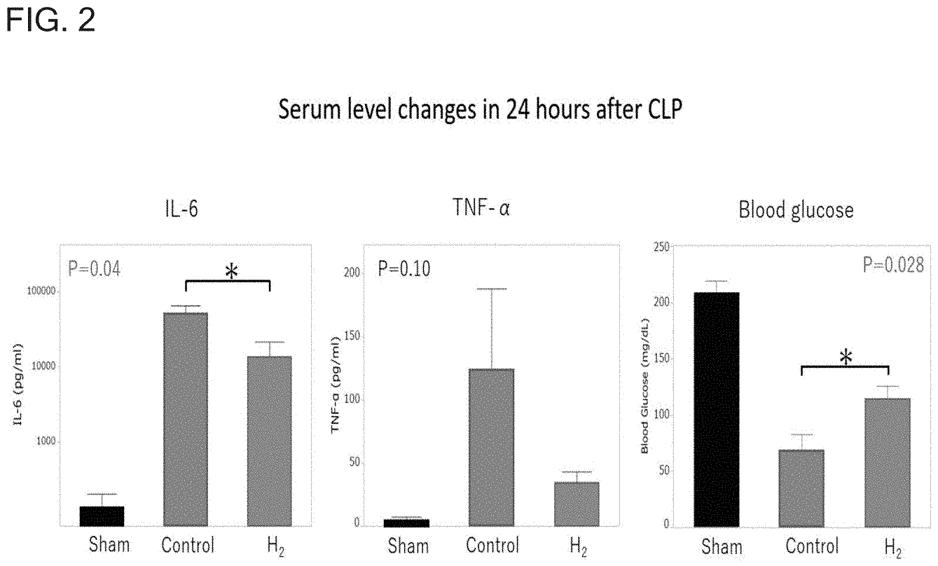

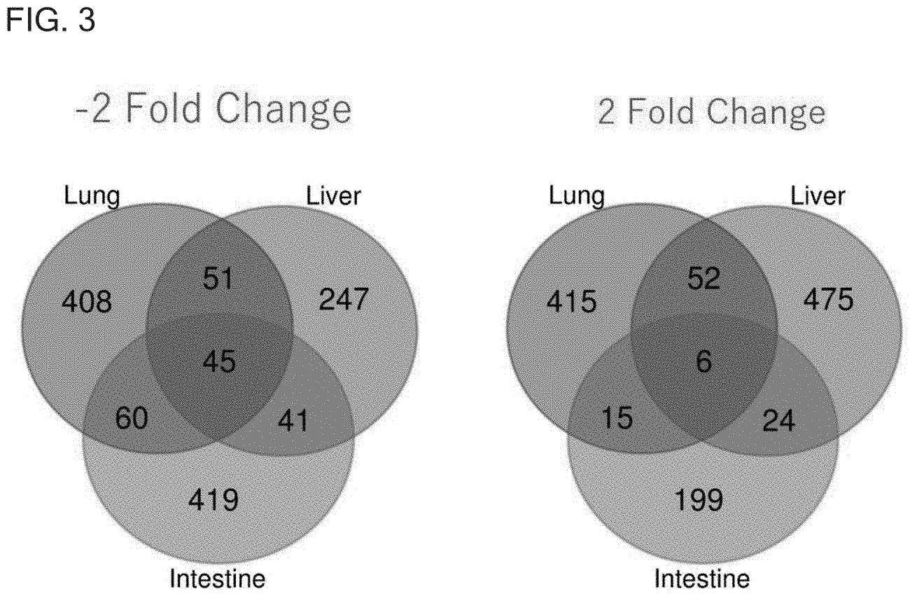

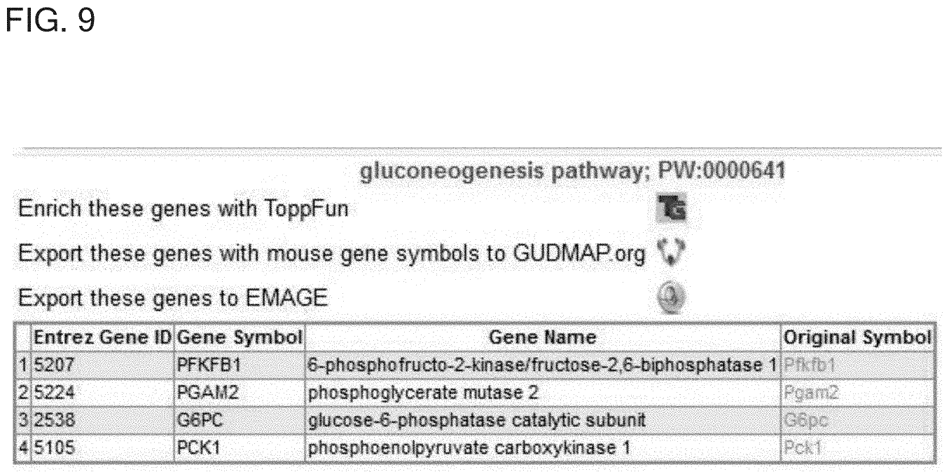

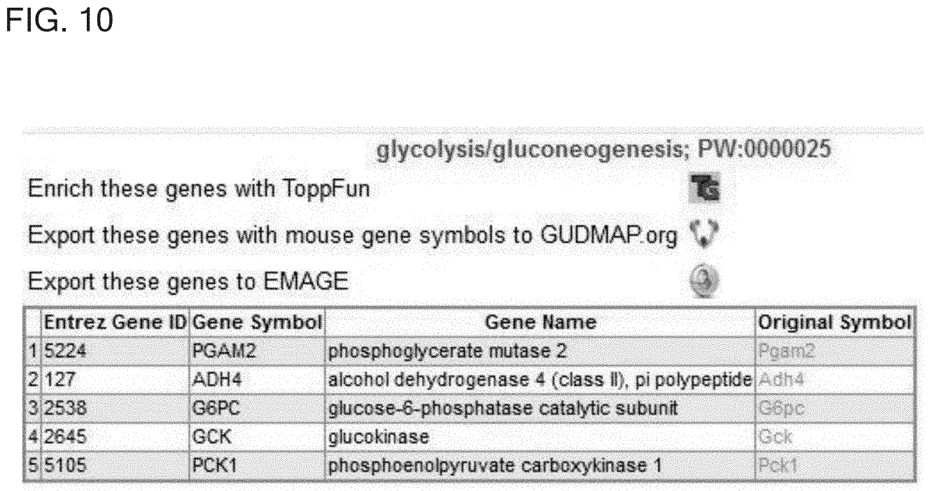

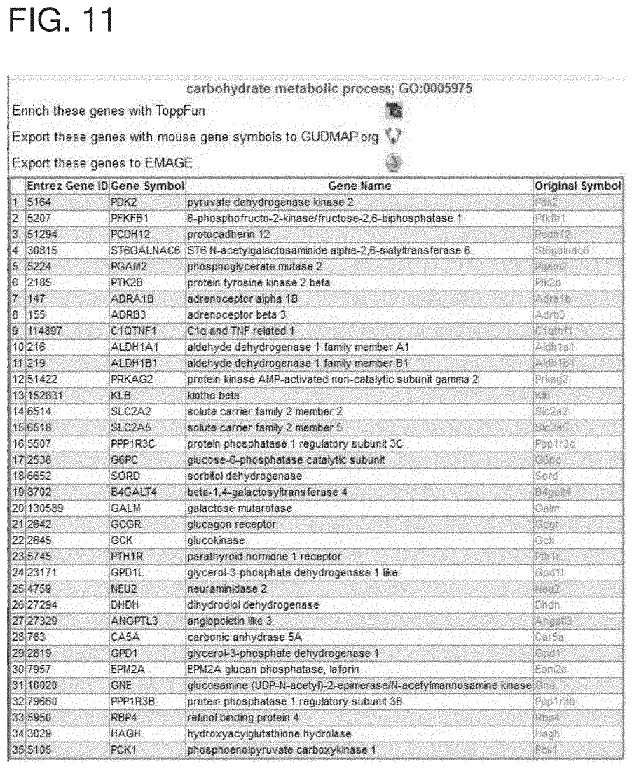

Treatment with a hydrogen gas attenuated the serum IL-6 and TNF-α levels at 24 hours after CLP, and improvement of blood glucose levels could be confirmed in the hydrogen gas treatment group (127 mg/dL vs. 74 mg/dL) [FIG. 2]. Given that GO and pathway enrichment analysis in the liver confirmed upregulation of genes related to glycolysis and gluconeogenesis, and the enrichment analysis confirmed a gene related to blood glucose production was significantly enhanced [FIG. 9 to FIG. 11], these results indicate that gluconeogenesis was induced by inhalation of hydrogen, and fatal hypoglycemia after 24 hours could be prevented.

When the hydrogen inhalation time was two hours or longer per day, differences started to be observed in the change in levels of these genes, and changes in the gene levels in the hydrogen group increased as the continuous inhalation time was increased to five hours, 12 hours, and 24 hours.

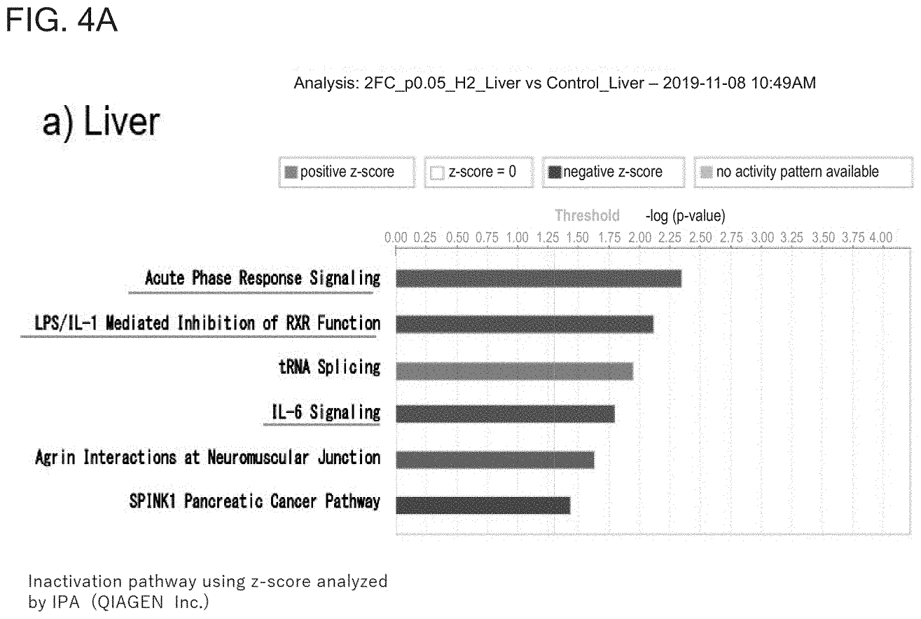

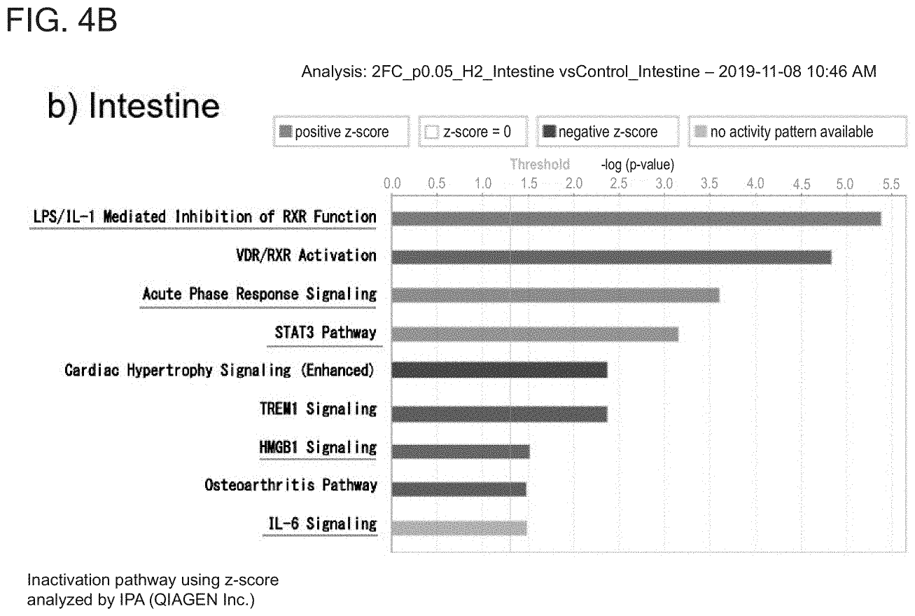

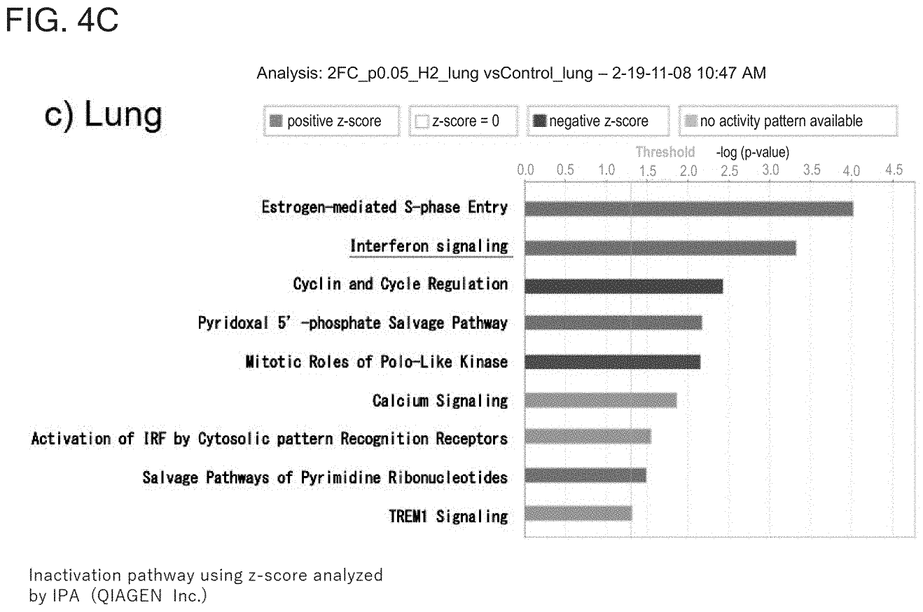

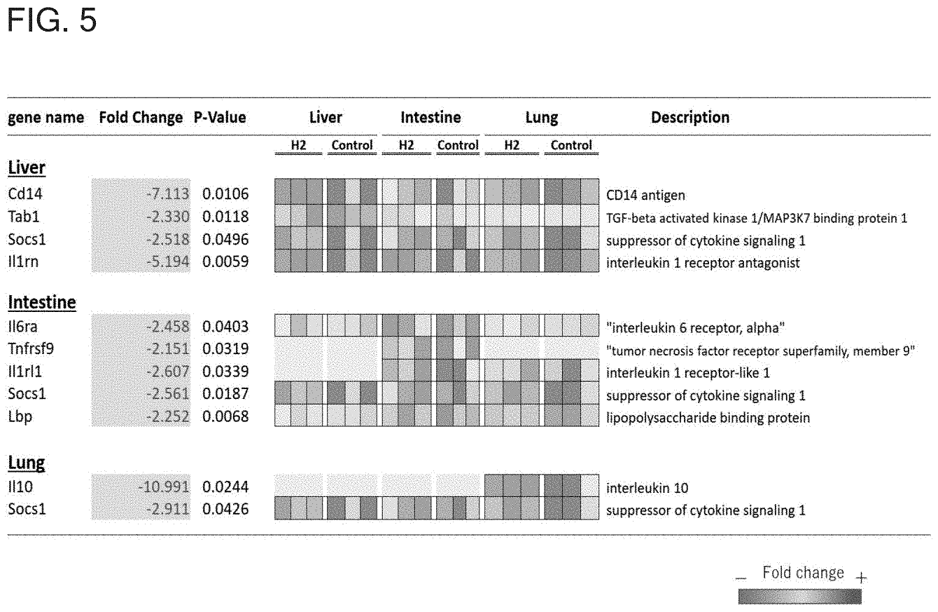

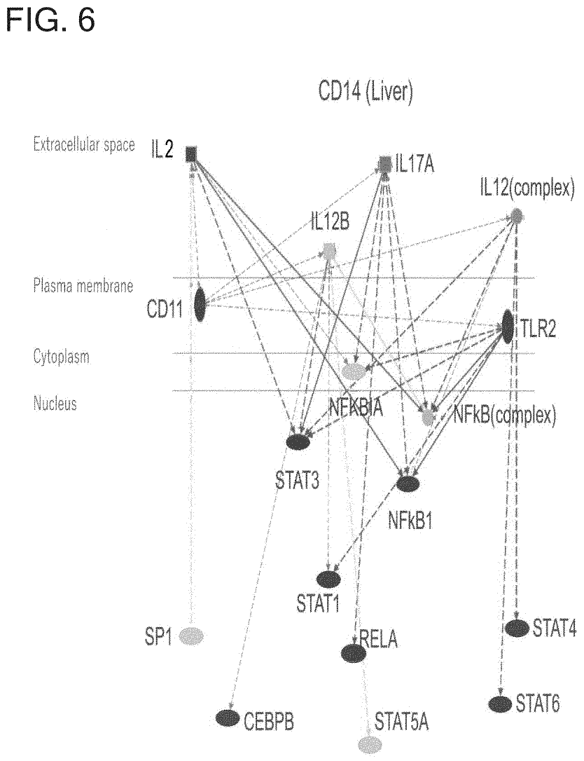

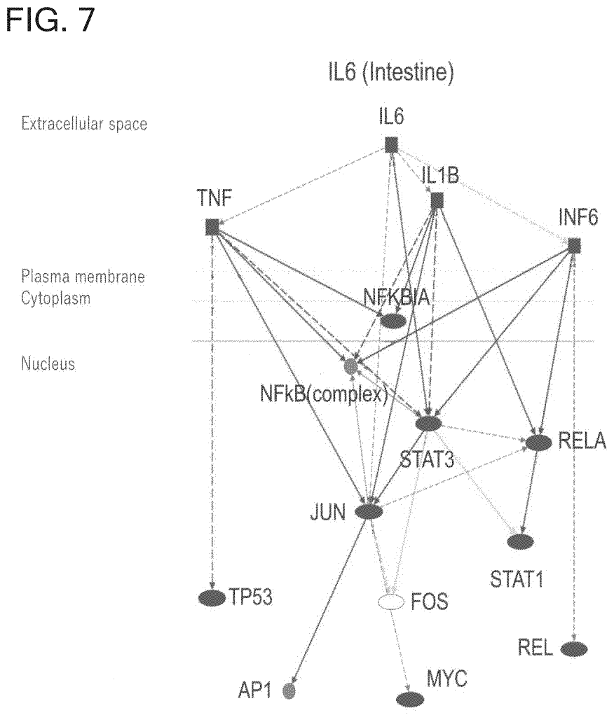

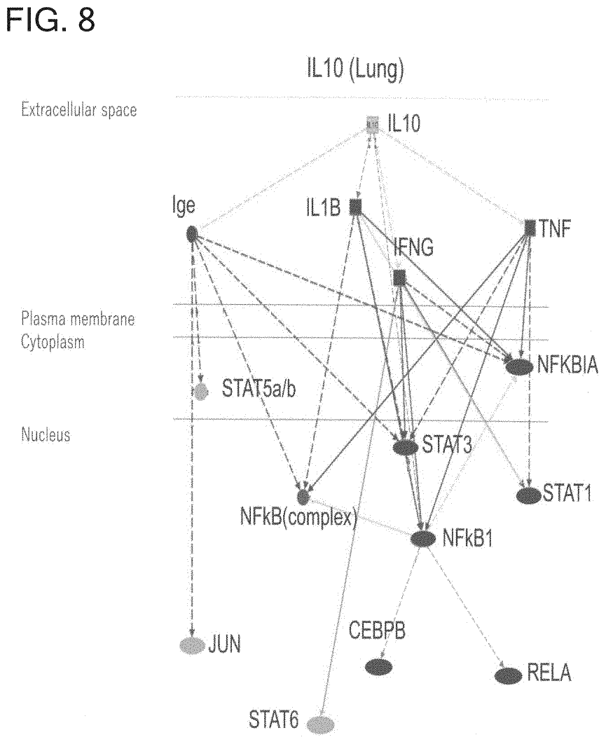

In the RNA sequencing, various inflammatory signal transduction pathways, such as acute phase response signaling and the STAT pathway, were found to be inactive in the liver and the intestine of the CLP model at 24 hours in the standard pathway analysis using z-scores [FIGS. 4A-4C]. The activities of transduction pathways of these inflammatory signaling became more inactive as the hydrogen inhalation time was increased.

Hydrogen was found to reach the intestine and blood vessels rapidly after oral administration and intraperitoneal administration of hydrogen water, and reach the lung rapidly and the liver after 10 minutes or longer after inhalation of a hydrogen gas. It was confirmed that administration of hydrogen water and intraperitoneal administration of hydrogen-containing physiological saline induced changes unique to intracellular signal transduction in the intestine, the liver, and the lung. Because intracellular signal transduction had been unknown before, administration of hydrogen effective for a subject could not be selected appropriately. However, by selecting any of inhalation of a hydrogen gas, oral administration of hydrogen water, and intraperitoneal administration of hydrogen-containing physiological saline by the present invention depending on the severity in the each of disease-related organ, improvement of the disease could be controlled precisely by regulating intracellular signal transduction.

### INDUSTRIAL APPLICABILITY

The present invention can regulate intracellular signal transduction by administering a composition comprising molecular hydrogen and make a strategic treatment plan using hydrogen.

What is claimed is:

1. A method for regulating intracellular signal transduction, comprising administering a composition containing an effective amount of molecular hydrogen.

2. The method according to claim 1, wherein the intracellular signal transduction is glycolysis, gluconeogenesis, and/or carbohydrate metabolism, and/or inflammatory signal transduction in an organ.

3. The method according to claim 2, wherein a gene related to the gluconeogenesis is one or more genes selected from the group consisting of PFKB1, PGAM2, G6PC, and PCK1.

4. The method according to claim 2, wherein a gene related to the glycolysis and gluconeogenesis is one or more genes selected from the group consisting of PGAM2, ADH4, G6PC, GCK, and PCK1.

5. The method according to claim 2, wherein a gene related to the glycolysis and carbohydrate metabolism is one or more genes selected from the group consisting of PDK2, PFKB1, PCDH12, ST6GALNAC6, PGAM2, PTK2B, ADRA1B, ADRB3, C1QTNF, ALDH1A1, ALDH1B1, PRKAG2, KLB, SLC2A2, SLC2A5, PPP1R3C, G6PC, SORD, B4GALT4, GALM, GCGR, GCK, PTH1R, GPD1L, NEU2, DHDH, ANGPTL3, CA5A, GPD1, EPM2A, GNE, PPP1R3B, RBP4, HAGH, and PCK1.

6. The method according to claim 2, wherein the organ is at least one of liver, lung, and intestine.

7. The method according to claim 2, wherein a pathway of the inflammatory signal transduction is acute phase response signaling in a liver and/or an intestine, LPS/IL-1 mediated inhibition of RXR function in the liver and/or the intestine, IL-6 signaling in the liver and/or the intestine, STAT3 pathway in the intestine, HMGB1 signaling in the intestine, or interferon signaling in a lung.

8. The method according to claim 7, wherein a gene related to the inflammatory signal transduction pathway is one or more genes selected from the group consisting of genes coding for Cd14 antigen in the liver, TGF-beta activated kinase 1/MAP3K7 binding protein 1 in the liver, suppressor of cytokine signaling 1 in the liver, interleukin 1 receptor antagonist in the liver, interleukin 6 receptor alpha in the intestine, tumor necrosis factor receptor superfamily member 9 in the intestine, interleukin 1 receptor-like 1 in the intestine, suppressor of cytokine signaling 1 in the intestine, lipopolysaccharide binding protein in the intestine, interleukin 10 in the lung, and suppressor of cytokine Signaling 1 in the lung.

9. The method according to claim 8, wherein, in a transduction pathway in the liver involving the gene related to the inflammatory signal transduction pathway, a signal is transmitted from IL-2 and/or IL12 outside a cell, via CD11 and/or TLR2 in the plasma membrane of the cell, further via NFkBIA in the cytoplasm, and via NFkB, STAT3, NFkB1, STAT1, STAT4, STAT6, STAT6A, SP1, RCLA, and/or GEBPB in the nucleus of the cell.

10. The method according to claim 8, wherein, in a transduction pathway in the intestine involving the gene related to the inflammatory signal transduction pathway, a signal is transmitted from IL6, IL1B, TNF, and/or IFN6 outside a cell, via NFkBIA in the cytoplasm, and further via NFkB, STAT3, RELA, JUN, TP53, STAT1, FOS, REL, Ap1, and/or MYC in the nucleus of the cell.

11. The method according to claim 8, wherein, in a transduction pathway in the lung involving the gene related to the inflammatory signal transduction pathway, a signal is transmitted from IL10, IL15, Ige, TNF, and/or IFNG outside a cell, via STAT5a/b and/or NFkBIA in the cytoplasm, and further via STAT3, NFkB, STAT1, NFkB1, JUN, CEBPB, and/or RELA in the nucleus of the cell.

12. The method according to claim 1, wherein the composition is a gas composition or a liquid composition.

13. The method according to claim 12, wherein the gas composition has a hydrogen concentration of higher than zero (0) and not higher than 18.5% by volume.

14. The method according to claim 12, wherein the liquid composition has a hydrogen concentration of 3 ppm to 10 ppm.

15. The method according to claim 1, wherein the composition is administered to a mammalian subject.

16. The method according to claim 1, wherein the composition is administered to a human.

17. The method according to claim 1, wherein the composition is produced by using a hydrogen gas generating apparatus or a hydrogen water generating apparatus.

18. The method according to claim 1, wherein a disease controllable by the regulation of intracellular signal transduction is one or more diseases selected from the group consisting of sepsis, inflammatory bowel diseases (ulcerative colitis, Crohn's disease, intestinal Bechet's disease), pneumonia (bacterial pneumonia, viral pneumonia, fungal pneumonia, interstitial pneumonia, allergic pneumonia), COPD, hepatitis (viral hepatitis, alcoholic hepatitis, NAFLD, drug-induced hepatitis, autoimmune hepatitis), diabetes mellitus, and complications of diabetes mellitus.

19. The method according to claim 1, wherein, based on the intracellular signal transduction in each organ, the intracellular signal transduction is regulated by an administration method selected from the group consisting of inhalation of a hydrogen gas, oral administration of hydrogen water, and drip infusion and/or intraperitoneal administration of hydrogen-containing physiological saline.

20. A system for deriving a treatment method and/or a treatment strategy for preventing or improving the disease according to claim 18 using a composition comprising an effective amount of molecular hydrogen.
